SEQUENCE LISTING (1 sequence)

SEQ ID NO: 1 - protein, 331 aa, Saccharomyces cerevisiae
VRVAINGFGRIGRLVMRIALSRPNVEVVALNDPFITNDYAAYMFKYDSTHGRYAGEVSHDDKHIIVDGKKIATYQERDPANLPWGSSNVDIAIDSTGVFKELDTAQKHIDAGAKKVVITAPSSTAPMFVMGVNEEKYTSDLKIVSNASCTTNCLAPLAKVINDAFGIEEGLMTTVHSLTATQKTVDGPSHKDWRGGRTASGNIIPSSTGAAKAVGKVLPELQGKLTGMAFRVPTVDVSVVDLTVKLNKETTYDEIKKVVKAAAEGKLKGVLGYTEDAVVSSDFLGDSHSSIFDASAGIQLSPKFVKLVSWYDNEYGYSTRVVDLVEHVAKA

(19) United States
(12) Patent Application Publication
(10) Pub. No.: US 2005/0196809 A1
(43) Pub. Date: Sep. 8, 2005

(54) Identification and characterization of proteins using new database search modes

(76) Inventor: Neil L. Kelleher, Urbana, IL (US)

Correspondence Address: EVAN LAW GROUP LLC, 566 WEST ADAMS, SUITE 350, CHICAGO, IL, 60661, US

(21) Appl. No.: 10/794,431

(22) Filed: Mar. 5, 2004

(51) Int. Cl. G01N 33/53; G06F 19/00; G01N 33/48; G01N 33/50

(52) U.S. Cl. 435/7.1; 702/19; 436/86

(57) ABSTRACT

A method of selecting a set of candidate polypeptides for a sample polypeptide that includes a first refining of a collection of candidate polypeptides from differences in mass of fragments of the sample polypeptide produced by mass spectrometry and a second refining of the collection of candidate polypeptides from the absolute mass of the sample polypeptide and the absolute mass of the fragments.

### STATEMENT OF ACKNOWLEDGMENT OF GOVERNMENT SUPPORT

This invention was made with Government support from the National Science Foundation (grant # CHE-0134953) and from the National Institutes of Health (grant # GM 067193-01). The Government has certain rights in the invention.

### APPENDIX MATERIALS

The appendix contains duplicate copies of one compact disk that provides software and database files. The contents of the compact disk are hereby incorporated herein by reference.

### BACKGROUND

One of the objectives of molecular biology is to characterize the structure and biochemical activity of proteins that are encoded by gene sequences. To a significant extent, the structural characterization of proteins relies on determining the primary structure (amino acid sequence) of proteins as they are expressed under native cellular conditions. Once a protein is translated from mRNA, the primary structure of the protein is often modified through the action of enzymes. These modifications include the addition of a new moiety to the side chain of an amino acid residue, such as the addition of phosphate to a serine or proteolytic cleavage, such as removal of an initiator methionine or a signal sequence. Thus, the structural characterization of a protein includes both the linear organization of the amino acid sequence (as affected by alternative splicing and polymorphisms) and the presence of any modification that may arise within the sequence.

Toward this end, a major goal of proteomic research is to understand the detailed modifications that occur on proteins. Such information is critical not only for understanding the biological activity of proteins, but for the development of pharmaceutical agents that control cell proliferation and differentiation for processes related to human disease.

Mass spectrometry (MS) is an analytical technique that is used to identify unknown compounds, to quantify known compounds, and to ascertain the structure of molecules. A mass spectrometer is an instrument that measures the masses of ions that have been converted from individual molecules. This instrument measures the molecular mass indirectly, in terms of a particular mass-to-charge ratio of the ions. The charge on an ion is denoted by the fundamental unit of charge of an electron z, and the mass-to-charge ratio m/z. Typically, the ions encountered in mass spectrometry have just a single charge (z=1) so the m/z value is numerically equal to the molecular mass in Da. For singly-charged ions, the m/z ratio is the mass of a particular ion.

Generally, MS bombards ions of a sample with high intensity photons, electrons or neutral gas, breaking bonds, resulting in the formation of fragment ions from the molecular ions of the intact molecule. Although both positive and negative ions are generated with MS, only one polarity of an ion is detected with a particular instrumental set-up. Formation of gas phase sample ions allows the sorting of individual ions according to mass and their detection. The sample, which may be a solid, liquid, or vapor, enters the vacuum chamber of the instrument through an inlet. Electrostatic and/or magnetic filters are used to sort the ions according to their respective m/z ratios, which are focused on the detector. In the detector, the ion flux is converted to a proportional electrical current. The instrument then records the magnitude of these electrical signals as a function of m/z and converts this information into a mass spectrum.

Absolute mass searching allows the unambiguous identification of a protein from a sequence database using the intact mass in combination with the mass of fragment ions (see FIG. 1). Identification is achieved by selecting all sequences from an annotated database that are within a user specified tolerance of an observed average or monoisotopic intact mass. Preferably, the candidate proteins are retrieved from a database of protein forms indexed by mass.

Each candidate sequence is then scored using the observed fragment ions. This process involves calculating all theoretical b/y or c/z• type fragment ion masses (average or monoisotopic) from each candidate sequence and counting the number of observed fragment ions that are within a user specified tolerance (absolute or part per million) of any theoretical fragment ion. The number of observed fragment ions and the number of observed fragment ions that correspond to theoretical fragment ions are used to calculate the probability that the identification is spurious. All calculated scores are multiplied by the number of candidate sequences considered to yield a probability-based score. The candidate protein with the lowest score (and thus the lowest probability of being a spurious identification) is then considered the most likely candidate protein.

MS has been used to determine the primary amino acid sequence of proteins. The mass differences observed for protein fragment ions may be used to deduce the amino acid composition of a portion of the protein sequence. These sequence tags may be used to identify the protein sequence, provided that MS data is available for a sufficient number of related protein fragment ions.

Strategies that use MS are now under development to improve the efficiency and reliability of detecting modifications of proteins on a proteomic scale. Although far fewer genes exist in mammalian genomes than once thought (Lander et al., 2001), alternate protein forms are possible for each gene as a consequence of nucleotide polymorphisms, alternative RNA splicing, RNA editing and post-translational modifications. In addition to regulating protein function by modification, environmental signals also lead to chemical modification of proteins. The detection of modifications presents a major opportunity for understanding the fundamental regulatory mechanisms of eukaryotic cells and for diagnosing human disease.

The most popular form of MS-based protein structure determination involves the use of a “bottom up” approach: an intact protein is initially digested with proteases of known specificity to generate shorter polypeptide fragments (see FIG. 2). These fragments are then purified and characterized using MS. Based upon the absolute mass observed for individual polypeptide fragments, the amino acid compositions may be inferred, and the identity of the protein can be deduced using searching algorithms and databases of known protein compositions. Using this approach, detection of modifications has been routinely performed on single proteins to generate peptide maps approaching nearly 100% sequence coverage (Biemann and Papayannopoulos, 1997). Yet this approach can leave gaps in the characterization of modifications since protease-derived fragments may undergo additional chemical changes and therefore not afford adequate redundant information on the original protein. Searching algorithms for this approach now support some type of detection and localization of modifications and are commonly available (Clauser et al., 1999; Perkins et al., 1999; Wilkins et al., 1999; and Zhang et al., 2000).

Measurement techniques are being developed to target modifications directly that are based on an analysis of peptide fragments derived from digestion of intact proteins with the protease trypsin. For example, detection of phosphorylation and glycosylation has been enhanced using various procedures, such as the isolation of modification-containing polypeptide fragments (e.g., based on the selective purification of modified peptides), the use of MS to detect a specific modification (e.g., scanning for marker ions of modified peptides) or with both methods (Goshe et al., 2001; Oda et al., 2001; Steen et al., 2001; Zhou et al., 2001; Ficarro et al., 2002). Finally, the bottom up approach has been used to detect differences in the modification profiles for proteins derived from two biological samples (e.g., phosphoproteomics) (Oda et al., 1999; Goshe et al., 2001; Oda et al. 2001; Zhou et al., 2001; Ficarro et al., 2002; Gerber et al., 2002). While some of these techniques are being scaled up for analysis of hundreds of proteins, none is general for all types of modifications.

An alternative approach, termed “top down,” has been developed to identify and characterize modifications in intact proteins (see FIG. 2). This approach uses tandem mass spectrometry (MS/MS or (MS)ⁿ) to first fragment the intact protein, and the fragments are then collected and subjected to subsequent rounds fragmentation and mass measurement. The top down approach therefore determines both the absolute mass of the intact protein and protein fragment ions. Since intact proteins are subject to MS, no structural information is inadvertently lost from the analysis; therefore, the top down approach has the potential to identify all modifications that occur within intact proteins. The top down approach has been used to obtain modification information for 32 proteins from as many as 4 organisms (Kelleher et al., 1998; Pineda et al., 2000; Reid et al., 2002; Meng et al., 2001).

The top down approach is general for all modifications. Modifications that have been characterized by the top down approaches to date include glycosylation (Reid et al., 2002; Ge et al., 2003), Cys alkylation (Kelleher et al., 1995), disulfide bond formation (Ge et al., 2002), oxidation (Ge et al., 2003), and phosphorylation (Meng et al., 2001). Major barriers to this approach are being lowered by improvements in protein purification procedures (Kachman et al., 2002; Meng et al., 2002), automation of Fourier transform MS (FTMS) (Johnson et al., 2002), development of quadrupole-FTMS hybrid instruments (Belov et al., 2001), and improvement of software necessary for the identification of intact proteins from MS/MS data (Reid et al., 2002; Meng et al., 2001). However, significant barriers still exist concerning data processing and retrieval software for the full characterization of proteins with modifications.

### SUMMARY

In one aspect, the present invention is a method of selecting a set of candidate polypeptides for a sample polypeptide that includes a first refining of a collection of candidate polypeptides from differences in mass of fragments of the sample polypeptide produced by mass spectrometry and a second refining of the collection of candidate polypeptides from the absolute mass of the sample polypeptide and the absolute mass of the fragments.

In a second aspect, the present invention is a computer program product for use with a computer. The computer program product includes a computer usable medium having computer readable program code in said medium for selecting a set of candidate polypeptides for a sample polypeptide. The computer program product includes computer readable program code for directing the computer to select a set of candidate polypeptides for a sample polypeptide that includes a first refining of a collection of candidate polypeptides from differences in mass of fragments of the sample polypeptide produced by mass spectrometry and a second refining of the collection of candidate polypeptides from the absolute mass of the sample polypeptide and the absolute mass of the fragments.

In a third aspect, the present invention is a system for selecting a set of candidate polypeptides for a sample polypeptide that includes means for a first refining of a collection of candidate polypeptides from differences in mass of fragments of the sample polypeptide produced by mass spectrometry, means for a second refining of the collection of candidate polypeptides from the absolute mass of the sample polypeptide and fragments of the sample polypeptide produced by mass spectrometry, and a computer.

Definitions

The term “fragments” and “fragment ions” are used interchangeably throughout the specification when referring to fragments of an intact polypeptide generated by mass spectrometry.

The term “nascent polypeptide” refers to the initial translation product of a mRNA.

The term “modification,” as used herein, refers to any chemical change in the primary structure of a nascent polypeptide. “Modification” of a protein includes: (i) a polymorphism at a codon position that results in a different amino acid within the primary structure of the protein; (ii) alternative splicing or RNA editing of a mRNA transcript that results in a different primary structure of a protein upon translation of the spliced or edited mRNA; and (iii) a chemical modification of the protein following its translation that results in a change in the molecular mass of the protein. Chemical modifications include naturally-occurring post-translational modifications as they arise in cells (e.g., proteolytic cleavage, protein splicing, N-Met and signal sequence removal, ribosylation, phosphorylation, alkylation, hydroxylation, glycosylation, oxidation, reduction, myristylation, biotinylation, ubiquination, iodination, nitrosylation, amination, sulfur addition, peptide ligation, cyclization, nucleotide addition, fatty acid addition, acylation, etc.) as well as modifications that occur from sources not endogenous to biological cells (e.g., environmental mutagens, chemical carcinogens, experimentally-induced artifactual modifications, etc.).

The phrase “shotgun annotation” refers to the description of a particular modification that occurs for an amino acid residue in a polypeptide (e.g., phosphorylation of the hydroxyl group of serine). Typically, the shotgun annotation may define a particular modification of an amino acid residue in a polypeptide that occurs within a defined sequence context (e.g., phosphorylation of the hydroxyl group of serine or threonine in the sequence: RXXS/TXRX, where X is any amino acid). Shotgun annotations result in the expansion of database to include protein forms that contain the designated modifications. Shotgun annotation includes any type of modification, as the term “modification” is used herein.

The phrase “dynamically modify” refers to creating a change to a software program or database during the performance of a search.

The phrase “dynamic shotgun annotation” refers to creating shotgun annotations to protein structures in a database during the performance of a search.

The term “expanding” refers to an increase in the number of protein forms in a collection following shotgun annotation of a smaller collection.

The phrase “expanded collection” refers to a collection of protein forms obtained following shotgun annotation of a smaller collection.

The term “refining” refers to a reduction in the number of protein forms in a collection following a query of a larger collection using either a sequence tag mode search or an absolute mass mode search.

The phrase “refined collection” refers to a collection of protein forms obtained following a query of a larger collection using either a sequence tag mode search or an absolute mass mode search.

The term “peptide” as used herein refers to a compound made up of a single chain of D- or L-amino acids or a mixture of D- and L-amino acids joined by peptide bonds. Preferably, peptides contain at least two amino acid residues and are less than about 50 amino acids in length.

“Polypeptide” as used herein refers to a polymer of at least two amino acid residues and which contains one or more peptide bonds. “Polypeptide” encompasses peptides and proteins, regardless of whether the polypeptide has a well-defined conformation. Preferably, a polypeptide is a naturally-occurring protein.

The term “protein” as used herein refers to a compound that is composed of linearly arranged amino acids linked by peptide bonds, but in contrast to peptides, has a well-defined conformation. Proteins, as opposed to peptides, preferably contain chains of 50 or more amino acids. Although proteins are referred throughout in the text, it is generally understood that the invention is applicable to all polypeptides.

The phrase “protein form” refers to a single species of a polypeptide or protein, including any modification. Thus, a single gene may encode many protein forms, depending upon the structure of the gene, the structure of the transcribed mRNA(s), and the nature of any modification(s).

The phrase “RNA splicing” refers to the removal of at least one intervening sequence of RNA by phosphodiester bond cleavage of two non-contiguous phosphodiester bonds within a given RNA and the joining the flanking exon RNA sequences by phosphodiester bond ligation.

The phrase “RNA editing” refers to an alteration in the nucleotide composition of an RNA sequence wherein at least one nucleobase of the transcribed RNA is replaced by another nucleobase of a different hydgrogen bonding specificity. The resultant edited RNA may encode for a polymorphism, an extended polypeptide sequence (e.g., by eliminating a stop codon or by introducing an initiator codon), or a truncated polypeptide sequence (e.g., by introducing a stop codon).

The phrase “RNA processing” refers to any reaction that results in covalent modification of an RNA sequence. “RNA processing” encompasses both RNA splicing and RNA editing.

The phrase “searching mode” refers to the process of identifying and retrieving candidate protein forms from a warehouse database.

The phrase “sequence tag” refers to a short terminal sequence of at least two contiguous amino acids for a fragment of a polypeptide that may be inferred from differences in mass of two related fragments of the polypeptide produced by mass spectrometry.

“Structure” as used herein with regard to proteins refers to the primary amino acid sequence of a protein, including modifications. The term “structure” and the phrase “primary structure” have the same meaning as used herein.

The phrase “warehouse database” refers to a collection of two or more protein forms.

### BRIEF DESCRIPTION OF THE DRAWINGS

FIG. 1 is a flow chart of the architecture that depicts the absolute mass mode searching procedure with MS data to obtain candidate proteins;

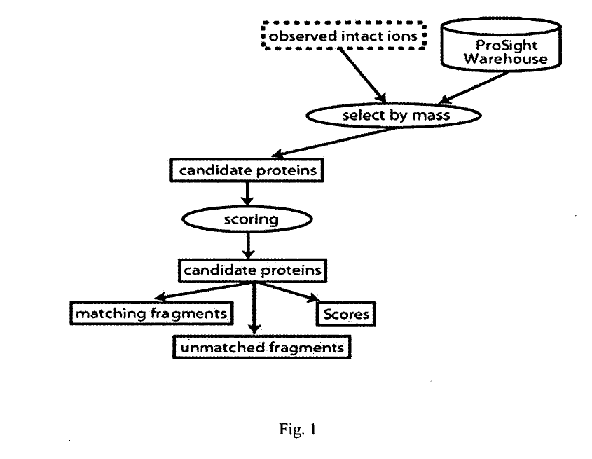

FIG. 2 illustrates the “top down” and “bottom up” approaches for protein identification and characterization of proteins by MS, wherein a modification (e.g., a post-translational modification (“PTM”)) may be identified and located;

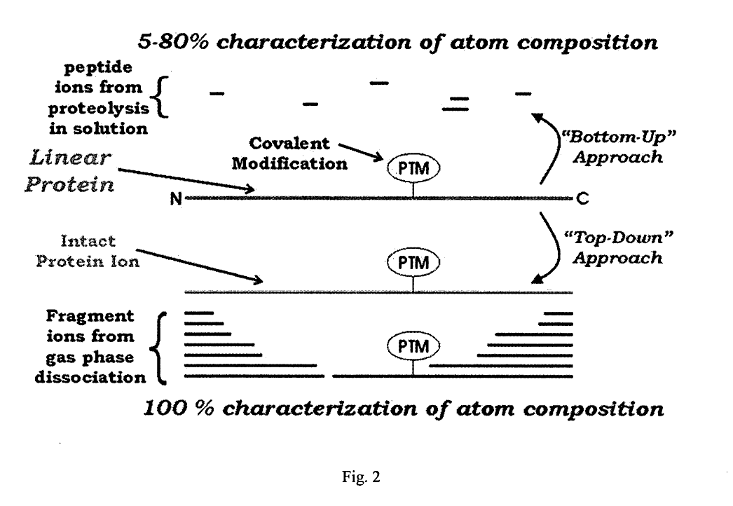

FIG. 3 depicts a process flow chart for the hybrid search mode methodology;

FIG. 4 is a flow chart of the software system that includes a retrieval algorithm (ProSight Retriever), a warehouse database of protein forms (ProSight PTM Warehouse) and primary utilities;

FIG. 5 depicts an embodiment where the databases are searched in “Delta m” mode;

FIG. 6 illustrates a schematic representation of shotgun annotation; and

FIG. 7 depicts an example of MS/MS for an ALS-PAGE/RPLC fraction from S. cerevisiae.

### DETAILED DESCRIPTION

The present invention makes use of the discovery of a hybrid searching mode methodology and software platforms to determine protein structure, including modifications. Hybrid searching mode methodology for determining the structure of proteins containing modifications uses a combination of one sequence tag mode search and one or more absolute mass mode searches to select a refined set of candidate polypeptides for a sample polypeptide. This methodology and associated software platforms are described below.

Hybrid Searching Mode Methodology

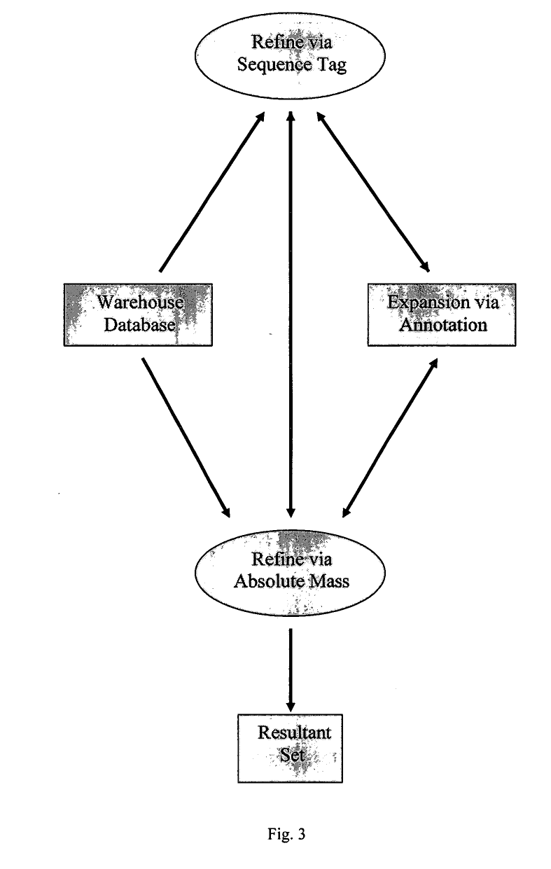

The hybrid search mode combines the sequence identification power of the sequence tag search with the modification detection and characterization power of the absolute mass search (see FIG. 3). This hybrid approach represents a more efficient method of refining collections of proteins than previously possible using either sequence tag or absolute mass searching protocols alone. In the hybrid search, sequence tags are compiled from the fragmentation data and a set of candidate proteins. The candidate proteins may originate from a warehouse database. The identity of each modification and its location within the protein is then determined using the absolute mass approach that focuses on the mass of the intact protein ion and the fragment ions. Any masses that are not accounted for in the theoretical mass of the protein form are usually attributable to the presence of modifications within the intact protein or protein fragment.

Preferably, a database of protein forms is initially populated with a large collection of proteins. Preferably, the initial database contains unannotated sequence information. Preferably, this database forms the initial collection of candidate polypeptides. In the preferred embodiment, the sequence tag search will refine a collection of candidate proteins that are composed of unmodified polypeptides. Optionally, the collection of candidate proteins may then be expanded with annotations of the candidate polypeptides to consider modifications. Preferably, following the sequence tag search, an absolute mass mode search is conducted on this collection to obtain a final set of candidate polypeptides. If the refined set contains only one protein form, then the absolute mass searching mode uniquely identified the modifications in the protein.

The hybrid searching mode methodology always employs one sequence tag mode search, followed by at least one absolute mass mode search. Optionally, an absolute mass mode search may be conducted prior to the sequence tag mode search. For example, a “three stage” search may be performed using the hybrid searching mode. This approach would use an initial absolute mass of the fragments with relaxed search parameters (e.g., minimal consideration of modifications or a large mass accuracy tolerance or both) to identify a collection of candidate sequences, followed by sequence tag mode searching to refine the collection of candidate sequences. An absolute mass mode search is then performed to further refine the collection.

Software Platforms

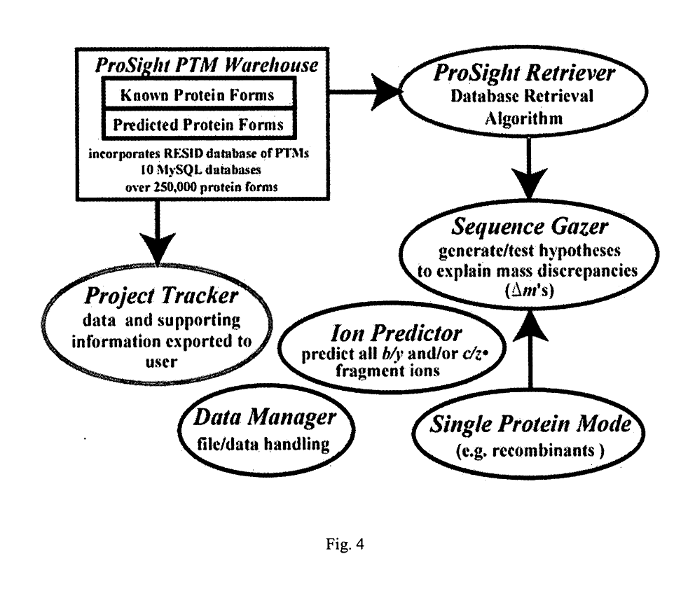

Computer software and systems are described that include a retrieval algorithm, a warehouse database of protein forms, and other utilities (see FIG. 4). The retrieval algorithm supports b/y and/or c/z• ion searches based on absolute mass values of observed fragment ions and sequence tag searches. The warehouse database of protein forms may include both unannotated and annotated modification information. Other utilities include a data management system, an ion predictor, a data reduction tool, and a graphical viewer interface tool.

Retrieval Algorithm

The retrieval algorithm facilitates top down identification of proteins including modification information by using a hybrid searching method that combines the sequence tag searching mode with the absolute mass searching mode. In reference to FIG. 3, one initially subjects MS data obtained for an intact protein and resultant protein fragment ions to a sequence tag search inquiry of a warehouse database of protein forms. In a sequence tag search, the user determines the partial sequence of the protein based upon the differences in mass of the fragment ions. Support of amino acids with the same nominal mass value (e.g., Ile and Leu; Lys and Gln) is provided when generating sequence tags. One implementation generates a graph representing all possible sequence tags that the data may contain. This graph is then analyzed to produce a regular expression for each represented sequence tag. One may then use this partial sequence information to select candidate proteins from a database of unannotated protein sequences. Optionally, the user may run a search with a manually compiled sequence tag set. Each candidate sequence receives a score calculated by multiplying the lengths of all sequence tags that match the sequence. For purposes of convenience, only sequences with a score higher than a specified tolerance are selected as data output.

Annotated sequence tags are generally not supported when searches are conducted with the sequence tag searching mode. This is reasonable, because it is unlikely that a sequence tag would overlap a site of modification and because the graphical representation of the data would become complicated with consideration that all possible modifications that may arise in a given collection of annotated sequence tags. Using this restriction, robust linear searches on protein databases can be implemented to obtain acceptable performance measurements for the retrieval functions (e.g., retrieval times are typically under three second running time for real queries).

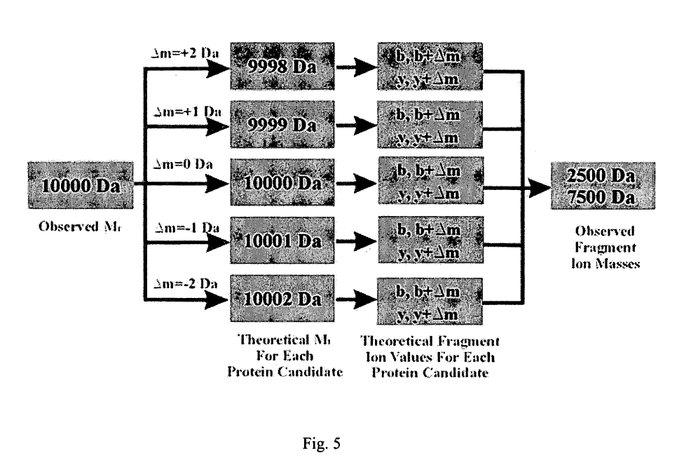

Optionally, an absolute mass search mode, termed delta M mode (“Δm mode”) allows one to search for proteins that harbor one modification of unknown identity or mass by considering the mass difference between the input intact MW value and the theoretical values housed in the database (see FIG. 5). A mass accuracy discrepancy can arise if a search is executed with an intact mass error of approximately ±1 Da. The accuracy of the Δm value is also ±1 Da, and the fragment ion mass accuracy can be a few parts-per-million (ppm). Depending on the chosen input settings, Δm values can be of varying accuracy.

Warehouse Database of Protein Forms

All identification algorithms using the top down approach initially select a collection of candidate sequences from a database. The unannotated forms of proteins are available as FASTA files on publicly accessible databases throughout the world, such as SWISS-PROT, GenBank, and the like. These databases may be mined to enable one to create the desired warehouse database of protein forms tailored for the particular project at hand. Preferably, PERL scripts are used to convert FASTA files to the files that are ready to populate the warehouse database While the FASTA file is converting, necessary information such as average and monoisotopic mass calculation and the number of amino acids in the sequence is added to the basic sequence from the FASTA file.

Shotgun Annotation of the Warehouse Database

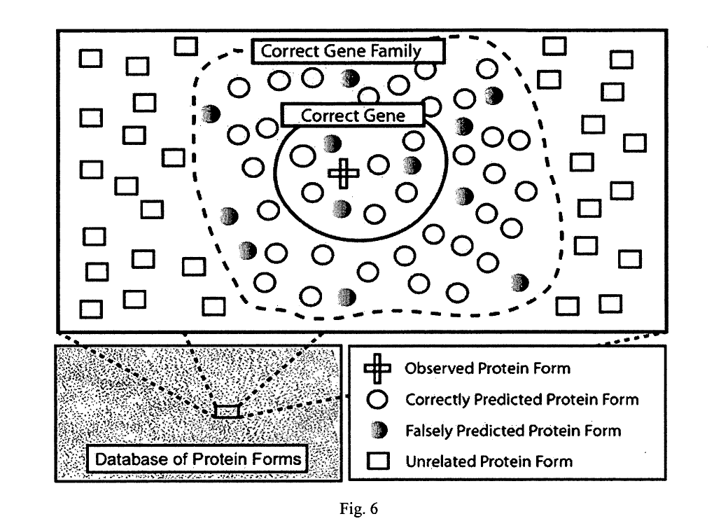

Given that the absence of the correct protein form in the database can hinder its identification, a data warehouse of annotated sequences is created using the nomenclature of RESID, which is an authoritative database of known modification types (Garavelli, 2003). Having a database of protein forms allows one to consider known and putative modifications that may be indicated by the occurrence of distinctive sequence motifs. This approach seeks to couple the partial or complete characterization of a protein form with its identification by retrieval of the known protein from a database of protein forms (see FIG. 6).

Post-translational modification events that may be annotated in the databases include N-terminal acetylation, signal peptide prediction, phosphorylation, lipoylation, GPI anchoring, ribosylation, alkylation, hydroxylation, glycosylation, oxidation, reduction, myristylation, biotinylation, ubiquination, nitrosylation, amination, sulfur addition, peptide ligation, cyclization, nucleotide addition, fatty acid addition, acylation, proteolytic cleavage, etc. (about 150-200 post-translational modifications are known for polypeptides (Garavelli, 2003) and may be considered as annotations). One can obtain modification annotations from publicly available databases, such as SWISS-PROT, or by manually entering the modification annotations into the warehouse database.

Preferably, each warehouse database has three tables that incorporate gene attributes, protein form attributes, and modification attributes. The gene attributes include gene identification information and a detailed description of the gene's structure. The protein form attributes include gene identification, protein form identification, monoisotopic mass, average mass, number of amino acids, and flags to any known attributes, such as a signal sequence, initiator Methionine, etc. The modification attributes include modification (RESID) identification, average mass, monoisotopic mass, and RESID code attributes.

The main job of the warehouse database is to handle the queries from the retrieval algorithm. Preferably, the retrieval algorithm always queries the warehouse database based on mass (either average or monoisotopic). Thus, the database should be indexed on mass and should return the corresponding sequences quickly so as not to decrease the speed of the entire system. The table of protein forms contains most of the information that the retrieval algorithm needs. Since the table of protein forms already contains all the annotated sequences and the masses, one may obtain rapid responses from the database to queries from the retrieval algorithm.

Although sites of modification may be theoretically predicted from the genetic sequence of the protein, it is often not desirable to populate the annotation database with all potentially possible annotations. The inclusion of such annotations will yield unwieldy databases from the standpoints of their shear size and of prolonged retrieval search times.

Once the retrieval algorithm identifies a refined collection of candidate proteins based upon the sequence tag search procedure, then one may generate an expanded collection containing all possible annotations for those particular proteins. This modification of the warehouse database does not compromise the performance of the retrieval algorithm because the searching inquiry is restricted to a small collection of possible protein forms. Therefore, a dynamic shotgun annotation of the warehouse database may be included in the hybrid searching approach. Once this collection protein candidates have been refined to yield a final set of candidate polypeptides and their associated modifications, the shotgun annotations that were entered dynamically into the warehouse database may be canceled before another sample polypeptide is characterized.

Ion Predictor

The ion predictor predicts a theoretical b/y and c/z ions, and is included in the software and system. Such calculations are useful for calculating errors, as expressed in terms of Daltons or parts-per-million (e.g., see Example 1, Table I).

Data Reduction Tool

A data reduction tool to remove redundant peaks resulting from multiple charge states and water/ammonia losses from reduced fragmentation data is included in the software and system. Such tools are useful for rapid analysis of the acquired MS data prior to its application by the retrieval algorithm.

Database Management System

Any database management system can be used with the warehouse database. Preferably, the database management system includes MySQL. The section of this popular database system is because it has many useful supporting tools and APIs, and the system is readily available to the public. The software provided in the appendix uses version 11.18 distribution 3.23.52 MySQL for Linux.

Graphical Viewer Interface Tool

In all search methods, a collection of candidate sequences is returned with varying scores. A graphical viewer interface tool for viewing a collection of candidate sequences derived from all searching approaches is included in the software and system. Optionally, the graphical viewer interface tool is incorporated into a local work station that includes the other features of the invention. Optionally, the graphical viewer interface tool is adapted for viewing data obtained via the internet from remote servers.

For the absolute mass mode search, the user is presented with the gene description, sequence, sequence length, theoretical mass, mass difference (absolute and ppm), the number of matching b (or c) type ions, the number of matching y (or z•) type ions, the total number of matching fragments, and the calculated probability score. The user may then sort the collection of candidate proteins by many of the listed headers and view fragmentation details for any retrieved sequence. The fragmentation details view presents the user with detailed information about every fragment that matches the sequence. This view presents the identified ion, the observed mass, the theoretical mass, the simple mass difference (i.e., before considering any mass shifts such as deduce through use of the “delta M” mode), and the mass difference shifted (i.e., after considering mass shifts as in “delta M” mode) and the shifted difference in parts per million. The graphical viewer interface tool also permits visualization of the fragmentation details, a feature useful for determining sequence coverage and spotting fragmentation patterns which increase user confidence in correct identification.

Databases Supported

The support databases can be configured for any organism. One embodiment supports databases for nine organisms, including: Saccharomyces cerevisiae, Escherichia coli, Arabidopsis thaliana, Bacillus subtilis, Methanococcus jannaschii, Mycoplasma pneumoniae, Shewanella oneidensis, Mus musculus and Homo sapiens. The yeast organism Saccharomyces cerevisiae database contains the most extensive annotations with known and predicted modification information.

Database Scalability

Of particular interest is how the database and search times scale with increasing modification information. A given gene and set of putative modifications results in an exponential number of protein forms where each form contains a subset of possible modifications. Thus, with n proteins and m possible processing events per protein, one embodiment includes a database containing O(n2ₘ) protein forms. Given that the retrieval search algorithm runs in O(mlog 2n) with the constant dependent upon the intact tolerance, the absolute mass search algorithm scales almost linearly with respect to m. With a database of known and putative protein forms, an observed protein form may be identified and characterized, provided that some modifications are correctly predicted. An increase of spurious information in publicly accessible protein databases will render ambiguous some searches based upon sparse MS/MS data. However, the number of matching fragment ion masses will increase with more extensive and accurate modification information used during the query step.

Computer Interface with Mass Spectrometry Instrumentation

Optionally the components are organized on a computer system in communication with a mass spectrometer. In one embodiment, the computer is a local work station. In another embodiment, the computer is a server located off-site. In the latter embodiment, the components may be stored on the server and accessed using internet-based interface tools. The MS data generated from the mass spectrometer is transmitted to the computer for data acquisition and storage. The computer's central processing unit coordinates analysis of the acquired MS data using the retrieval algorithm operating in one of the preferred embodiments to search the warehouse database of protein forms. Operator-specified tolerances are selected from options provided by the retrieval algorithm software to permit collection of protein candidates from the warehouse database of protein forms for further analysis of modifications.

Medical Applications

One can discern the effects of environmental signals on the extent of modification on particular target proteins in vivo. For example, many human disease conditions are regulated by modifications, such as phosphorylation. One may diagnose epigenetic disorders that are drawn to modification-based alterations of specific genes within families. Specific proteins can be surveyed for the presence of unusual modifications, and provide novel insight about disease states that might otherwise correlate poorly with alterations within known gene sequences. The system therefore provides a robust platform for screening disorders or individuals who have a predisposition to particular diseases.

Where modification alterations of individual proteins are implicated in the etiology of the disease, the system may be configured for use in the research setting to facilitate discovery of pharmaceutical compounds that control or modulate modification addition or removal to particular proteins. In one embodiment disclosed herein, the system is implemented as an integral component of a high throughput screening strategy wherein combinatorial libraries of candidate pharmaceutical compounds are evaluated for their ability to promote or inhibit an enzyme associated with modification activity to catalyze modification on a particular protein substrate. The protein substrate is interrogated for the presence (or absence) of the modification using MS. Compounds that possess the desired pharmaceutical effects may then be used in secondary tier drug development programs drawn to particular diseases.

The system may be configured for use in the clinical setting to evaluate the efficacy of pharmaceutical compounds that control or modulate modification addition or removal to particular proteins. In one embodiment, the system can be used to ascertain from patient samples whether specific proteins bear modifications in response to pharmaceutical treatment. For example, the target protein of interest may be purified to homogeneity from lysates prepared from patient samples and subjected to MS/MS analysis according to methods, software and system described herein. Differences between the MS data obtained for the sample protein relative to the corresponding protein form with all of its natural shotgun modification annotations contained in the warehouse database would be readily obtained and informative as to the pharmaceutical activity of the treatment regimen.

It will be readily apparent to one skilled in the field that the invention can be used to detect a variety of modifications in a protein regardless of their mechanism of occurrence. For example, one may use the invention to identify and characterize on a single protein the location of a polymorphism, the effect of RNA splicing or RNA editing of a mRNA on the resultant protein sequence, the presence of a post-translation modification, and an environmentally-induced chemical modification. Furthermore, it will be appreciated by one of ordinary skill that the hybrid mode search methodology permits detection of any biological event or bioinformatic imprecision that creates a mass discrepancy between the theoretically-predicted polypeptide form and the actually-measured polypeptide.

ProSight PTM: Software and Structure

The appendix contains a compact disk that provides all the necessary software tools and sample annotated warehouse database of protein forms to perform the disclosed aspects and embodiments. The system titled “ProSignt PTM” is a preferred embodiment. This system contains four main components, all with internet-based interfaces: a protein database (ProSight Warehouse), a database retrieval algorithm (Retriever), a data manager, a project tracker, and other utilities (see FIG. 4; Taylor et al., 2003).

Time-critical tasks, such as database retrieval and scoring, were written using an object-oriented design in C++ on Linux using the iODBC libraries for database connectivity. The data reduction tool is written in OCaml (chosen for language expressivity) while the visualization tool is written in PERL using the GD module for rendering images.

Use of the absolute mass search requires a running implementation of ProSight Warehouse on an ODBC enabled database management system. The internet application is written in PERL using CGI served by the Apache HTTP server running on a dual processor Athlon 2200+ MP.

### EXAMPLES

Several embodiments are disclosed with specific illustrations focused on MS/MS analysis of modifications associated with a S. cerevisiae 36-kDa protein, which was later identified as the Glyceraldehyde Phosphate Dehydrogenase Type 3 enzyme. Though Q-FTMS was used, data about intact proteins obtained from any type of mass spectrometer can substitute. A database strategy is described to use known and putative modification information for improved retrieval scores and modification characterization rates as desired for the particular application at hand.

### Example 1

### Automated Top Down Analysis of a Native Yeast Protein

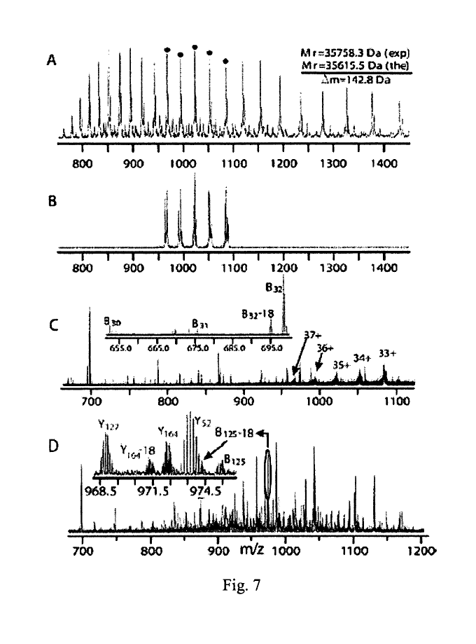

A yeast protein with a Mr value of 35,758.3 Da was observed in one ALS-PAGE/RPLC fraction (FIG. 7A). There are three other components in the same sample, with one of these corresponding to a phosphate adduct (+98 Da) attached to the 35.8-kDa species. The on-line deconvolution algorithm picked out the 35.8-kDa protein and generated an appropriate SWIFT waveform to select out the five charge states shown in FIG. 7B. Using the IR laser, the MS/MS spectrum of FIG. 7C was generated automatically with 39 isotopic distributions observed corresponding to 27 discrete fragment ion mass values automatically detected by the THRASH algorithm. After a filter to remove spurious peaks (e.g., water loss peaks), 20 ion masses were used as the final input for the database retrieval. This protein was identified to be glyceraldehyde-3-phosphate dehydrogenase (GAPDH3), with nine b-type ions and 3 y-type ions matched (Tables I and II). The P-score for this retrieval was 4×10⁻⁸, indicating that this identification was unlikely to be a spurious event.

TABLE I
Ion fragmentation data of GAPDH3 (SEQ ID NO: 1)¹

| | Observed | Theoretical | | |
|---|---|---|---|---|
| Ion | Mass (Da) | Mass (Da) | Error (Da) | Error (PPM) |
| B26 | 3072.81 | 3072.8 | 0.02 | 5 |
| B29 | 3143.85 | 3143.83 | 0.02 | 6 |
| B30 | 3256.91 | 3256.92 | 0 | −1 |
| B31 | 3370.98 | 3370.96 | 0.02 | 5 |
| B32 | 3486.01 | 3485.99 | 0.02 | 6 |
| B33 | 3583.06 | 3583.04 | 0.02 | 6 |
| B34 | 3730.12 | 3730.11 | 0.01 | 3 |
| B82 | 9227.73 | 9227.75 | −0.02 | −3 |
| B89 | 9955.03 | 9955.08 | −0.06 | −6 |
| Y52 | 5733.78 | 5733.83 | −0.05 | −9 |
| Y53 | 5832.82 | 5832.9 | −0.08 | −13 |
| Y139 | 14810.62 | 14810.81 | −0.19 | −13 |

¹GAPDH3 has 331 amino acids; theoretical mass of 35,615.5 Da; Δm 142.8 Da

TABLE II
Graphical Fragment Map of GAPDH3 (SEQ ID NO:1)¹

V R V A I N G F G R I G R L V M R I A L S R P N V
E V V┘A┘L┘N┘D┘P┘F┘I T N D Y A A Y M F K Y D S T H
G R Y A G E V S H D D K H I I V D G K K I A T Y Q
E R D P A N L┘P W G S S N V┘D I A I D S T G V F K
E L D T A Q K H I D A G A K K V V I T A P S S T A
P M F V M G V N E E K Y T S D L K I V S N A S C T
T N C L A P L A K V I N D A F G I E E G L M T T V
H S L T A T Q K T V D G P S H K D┌W R G G R T A S
G N I I P S S T G A A K A V G K V L P E L Q G K L
T G M A F R V P T V D V S V V D L T V K L N K E T
T Y D E I K K V V K A A A E G K L K G V L G Y T E
D A V┌V S┌S D F L G D S H S S I F D A S A G I Q L
S P K F V K L V S W Y D N E Y G Y S T R V V D L V
E H V A K A

¹The underlined Cys residues are those identified to contain an acrylamide modification. The symbol ┘ refers to amino-derived fragment ions while the symbol ┌ refers to carboxyl-derived fragment ions.

This gene product (GAPDH3; SEQ ID NO:1) was successfully distinguished from others in the GAPDH gene family, GAPDH2 (SEQ ID NO:2) and GAPDH1 (SEQ ID NO:3), with 96% and 80% sequence identity, respectively. These data also discerned this protein form from a conflict reported by ExPASy, with only 3 out of 331 amino acid residues different. Further, the observed molecular mass of the GAPDH3 gene product was 142 Da larger than the theoretical value calculated from the sequence in the database (no initiator Met). The fragment map localized this mass discrepancy (Δm) between Asp₉₀ and Asp₁₉₂, with the only two Cys residues (Cys₁₄₉ and Cys₁₅₃) in this sequence region (see Table II).

The subsequent interrogation of this protein form using manual Q-FTMS/MS and collisional dissociation of ions outside the superconducting magnet yielded the spectrum of FIG. 7D, with 98 isotopic distributions. Using these data as input into the retrieval algorithm further narrowed the +142 Da Δm to the Pro₁₂₆-Leu₁₅₄region. These data are consistent with the two Cys residues alkylated by acrylamide (+71 Da each) during gel electrophoresis. Though not localized exactly to Cys₁₄₉ and Cys₁₅₃, this in-gel modification has several precedents and is expected for free thiols in a PAGE-based fractionation. Thus, the overall process involved initial detection of covalent modifications using the top down approach.

Given that absolute mass retrieval times are linearly dependent upon the number of candidate sequences scored, smaller intact tolerances expedite retrieval time. A simple search of yeast with a ±2-kDa tolerance takes 6 s for 1500 candidates while the same search with a 200-Da tolerance completes in 400 ms for 200 candidates. Hybrid searches are linearly dependent upon number of FASTA file entries and the number of sequence tags considered. A search with five sequence tags completes in 4 s. Of the yeast proteins fragmented to date, approximately half can be identified using the absolute mass of observed fragment ions with the retrieval algorithm. For the remainder, 20% could be identified via the sequence tags generated from the relative mass difference between observed fragment ions. In sequence tag mode, automated compiling of the FIG. 7C data gave four tags (two real, two spurious, each of length 4 amino acids). Restricting the compilation of sequence tags to fragment ions of the same charge gave only the two correct tags. Using the data of FIG. 7D, five of eight tags were spurious (length: 1-4 amino acids) and four of six were spurious (length: 1-3 amino acids) with the charge-state restriction.

### Example 2

### Screening Compounds that Modulate an Enzyme with Modification Activity (Prophetic Example)

The purpose of the following example is to outline a high throughput strategy for identifying compounds from a combinatorial library that modulate in either a positive or negative manner the function of an enzyme that displays modification activity. Although the particular example is set forth in an in vitro environment, adaptations of the example to in vivo contexts are readily appreciated.

A recombinant form of the human Src kinase oncoprotein containing an N-terminal histidine tag (UpState Biotechnology, Inc.; Lake Placid, N.Y.) is immobilized onto 96-well dishes coated with Ni-NTA resins in Src kinase buffer (100 mM Tris-HCl (pH 7.2), 125 mM MgCl₂, 25 mM MnCl₂, 2 mM EGTA, 500 μM ATP, 0.25 mM sodium orthovanadate, and 2 mM dithiothreitol). After the addition of the test compounds in Src kinase buffer, preferably one homogeneous compound per well, a Src protein substrate of known sequence is added to each well (at a concentration of 100-300 μM) to permit its phosphorylation. Following incubation, the substrate is recovered and subjected to top down mass spectrometry using the ProSight PTM system.

The ability of a particular compound to inhibit Src activity will be discerned by the absence of a modification associated with a phosphorylated tyrosine reside within the protein. Such compounds are suitable for further characterization using other assays to confirm the top down analysis. For example, one may use [γ-³²P]ATP in assays and monitor phosphorylation activity using TCA precipitation assays on P81 paper.

### Example 3

### Detection of an Epigenetic Disorder in an Individual (Prophetic Example)

The purpose of this example is to demonstrate the utility of the ProSight PTM system for detecting modifications associated with an epigenetic disorder using top down mass spectrometry. Sample tissue is acquired from chickens infected with the avian sarcoma virus as well as from uninfected chickens. The samples is homogenized and clarified to produce a soluble lysate. The γ-catenin protein, a known in vivo substrate of the avian Src kinase, is affinity purified from the lysates using anti-γ-catenin antibody. The recovered γ-catenin samples will then be subjected to analysis using top down mass spectrometry and ProSight PTM. The expected results are that the γ-catenin protein recovered from normal tissues will display the normal modification profile of the protein form stored in the ProSight Warehouse database, whereas the γ-catenin protein recovered of infected chickens will include additional modifications associated with tyrosine phosphorylation.

### Example 4

### Experimental Procedures for Examples 1-3

Cell Culture and Lysate Fractionation

S. cerevisiae cells (strain S288C) were grown under aerobic conditions. Approximately 2 g of cells (wet mass) was resuspended in 10 mL of lysis buffer (25 mM Tris, 1 mM EDTA, 1 mM TCEP, pH 7.0, 1 mL of DNAase added), with two protease inhibitor tablets (Roche Diagnostics, Mannheim, Germany). After lysis by French press, the cellular debris was clarified by centrifugation for 30 min at 10,000×g. The supernatant was then mixed with acid-labile surfactant (ALS) sample buffer before loading on a model 491 preparative gel apparatus (Bio-Rad), with 0.1% ALS-I used instead of 0.1% SDS. A 4% T stacking gel was used with 12% T resolving gel eluted at a flow rate of 0.50 mL/min. Of the 80 fractions collected (2 mL each), 2 were processed further by cold acetone precipitation, resuspension in 6 M guanidine hydrochloride (pH 2), and subjected to reversed-phase liquid chromatography (RPLC) using a symmetry 300 C4 column (4.6×50 mm; Waters Inc., Milford, Mass.) with a linear gradient over 15 min using standard solvents (H₂O, CH₃CN, and 0.1% TFA).

ESI-Q-FTMS Instrumentation

RPLC-fractionated proteins were dried down and resuspended in 80 μL of ESI solution (50% ACN, 49% H₂O, and 1% formic acid) before being loaded into a nanospray robot (Advion BioSciences, Ithaca, N.Y.) for direct analysis of 5-10 μL samples at ˜100 nL/min. The 8.5-T Q-FTMS instrument used in this study was constructed in-house as described elsewhere. In short, protein ions were first stored in an octopole and then transferred through a quadrupole before accumulation in a second octopole before final analysis in the ICR cell. The quadrupole can be operated in either mass selection or “rf-only” mode. The automation script written in Tcl acquires a spectrum of intact proteins and then calls an on-line deconvolution algorithm to calculate the Mr values and SWIFT isolate the five most abundant charge states. After 5 scans for the isolated charge states, the IR laser is turned on for either 25 or 50 scans (0.45 s, 75% power, 40-W laser). The Q-FTMS/MS spectrum of FIG. 7D was acquired manually by collisional dissociation of specific charge states as they transfer from the quadrupole into a second octopole.

### REFERENCES

Belov M E, Nikolaev E N, Anderson G A, Auberry K J, Harkewicz R, Smith R D. “Electrospray ionization-Fourier transform ion cyclotron mass spectrometry using ion preselection and external accumulation for ultrahigh sensitivity,” J. Am. Soc. Mass Spectrom. 12:38-48 (2001).
Biemann K, Papayannopoulos I. Acc. Chem. Res. 27:370-78 (1994).
Clauser K R, Baker P, Burlingame A L. “Role of accurate mass measurement (+/−10 ppm) in protein identification strategies employing MS or MS/MS and database searching,” Anal. Chem. 71:2871-82 (1999).
Ficarro S, McCleland M, Stukenberg P, Burke D, Ross M, Shabanowitz J, Hunt D, White F. “Phosphoproteome analysis by mass spectrometry and its application to Saccharomyces cerevisiae,” Nat. Biotechnol. 20:301-305 (2002).
Garavelli, J S. “The RESID Database of Protein Modifications: 2003 developments,” Nucleic Acids Res. 31:499-501 (2003).
Ge Y, Lawhorn B G, ElNaggar M Strauss E, Park J H, Begley T P, McLafferty F W. “Top down characterization of larger proteins (45 kDa) by electron capture dissociation mass spectrometry,” J. Am. Chem. Soc. 124:672-78 (2002).
Ge Y, ElNaggar M, Sze S K, Bin O H, Begley T P, McLafferty F W, Boshoff H, Barry C E. J. Am. Soc. Mass Spectrom. 14:253-61 (2003).
Gerber S A, Rush J, Stemmann O, Steen H, Kirschner M W, Gygi S P. In: 50th ASMS Conference on Mass Spectrometry and Allied Topics, Orlando, Fla., 2002.
Goshe M B, Conrads T P, Panisko E A, Angell N H, Veenstra T D, Smith R D. “Phosphoprotein isotope-coded affinity tag approach for isolating and quantitating phosphopeptides in proteome-wide analyses,” Anal. Chem. 2001, 73:2578-86 (2001).
Johnson J R, Meng F, Forbes A J, Cargile B J, Kelleher N L. “Fourier-transform mass spectrometry for automated fragmentation and identification of 5-20 kDa proteins in mixtures,” Electrophoresis 23:3217-23 (2002).
Kachman M T Wang H, Schwartz D R, Cho K R, Lubman D M. “A 2-D liquid separations/mass mapping method for interlysate comparison of ovarian cancers,” Anal. Chem. 74:1779-91 (2002).
Kelleher N L, Costello C A, Begley T P, McLafferty F W. J. Am. Soc. Mass Spectrom. 6:981-84 (1995).
Kelleher N L, Taylor S V, Grannis D, Kinsland C, Chiu H J, Begley T P, McLafferty F W. “Efficient sequence analysis of the six gene products (7-74 kDa) from the Escherichia coli thiamin biosynthetic operon by tandem high-resolution mass spectrometry,” Protein Sci. 7:1796-1801 (1998).
Lander E S et al. “Initial sequencing and analysis of the human genome,” Nature 409:860-921 (2001).
MacCoss M J McDonald W H, Saraf A, Sadygov R, Clark J M, Tasto J J, Gould K L, Wolters D, Washburn M, Weiss A Clark J I, Yates J R., III. “Shotgun identification of protein modifications from protein complexes and lens tissue,” Proc. Natl. Acad. Sci. U.S.A. 99:7900-7905 (2002).
Meng F, Cargile B J, Miller L M, Forbes A J, Johnson J R, Kelleher N L. “Informatics and multiplexing of intact protein identification in bacteria and the archaea,” Nat. Biotechnol. 19:952-57 (2001).
Meng F, Cargile B J, Patrie S M, Johnson J R, McLoughlin S M, Kelleher N L. “Processing complex mixtures of intact proteins for direct analysis by mass spectrometry,” Anal. Chem. 74:2923-29 (2002).
Oda Y, Huang K, Cross F R, Cowburn D, Chait B J, “Accurate quantitation of protein expression and site-specific phosphorylation,” Proc. Natl. Acad. Sci. U.S.A. 96:6591-96 (1999).
Oda Y, Nagasu T, Chait B T. “Enrichment analysis of phosphorylated proteins as a tool for probing the phosphoproteome,” Nat. Biotechnol. 19:379-82 (2001).
Perkins D, Pappin D, Creasy D, Cottrell J. “Probability-based protein identification by searching sequence databases using mass spectrometry data,” Electrophoresis 20:3551-67 (1999).
Pineda F J, Lin J S, Fenselau C, Demirev P A. “Testing the significance of microorganism identification by mass spectrometry and proteome database search,” Anal. Chem. 72:3739-44 (2000).
Reid G E, Shang H, Hogan J M, Lee G U, McLuckey S A. “Gas-phase concentration, purification, and identification of whole proteins from complex mixtures,” J. Am. Chem. Soc. 124:7353-62 (2002).
Reid G E, Stephenson J L, McLuckey S A. “Tandem mass spectrometry of ribonuclease A and B: N-linked glycosylation site analysis of whole protein ions,” Anal. Chem. 74:577-83 (2002).
Steen H, Kuster B, Fernandez M, Pandey A, Mann M. “Detection of tyrosine phosphorylated peptides by precursor ion scanning quadrupole TOF mass spectrometry in positive ion mode,” Anal. Chem. 73:1440-48 (2001).
Taylor G K, Kim Y B, Forbes A J, Meng F, McCarthy R, Kelleher N L “Web and database software for identification of intact proteins using top down mass spectrometry,” Anal. Chem. 75:4081-86 (2003).
Wilkins M R, Gasteiger E, Gooley A A, Herbert B R, Molloy M P, Binz P A, Ou K, Sanchez J C, Bairoch A, Williams K L, Hochstrasser D F. “High-throughput mass spectrometric discovery of protein post-translational modifications,” J. Mol. Biol. 289:645-57 (1999).
Zhang W, Chait B. “ProFound: an expert system for protein identification using mass spectrometric peptide mapping information,” Anal. Chem. 72:2482-89 (2000).
Zhou H, Watts J D, Aebersold R. “A systematic approach to the analysis of protein phosphorylation,” Nat. Biotechnol. 19:375-78 (2001).

1. A method of selecting a set of candidate polypeptides for a sample polypeptide, comprising:
a first refining of a collection of candidate polypeptides from differences in mass of fragments of the sample polypeptide produced by mass spectrometry; and
a second refining of the collection of candidate polypeptides from the absolute mass of the sample polypeptide and the absolute mass of the fragments.

2. The method of claim 1, wherein the first refining comprises determining at least a partial amino acid sequence of the sample polypeptide from the differences in mass of the fragments.

3. The method of claim 2, further comprising:
determining the absolute mass of an intact form of the sample polypeptide and the absolute mass of the fragments of the sample polypeptide.

4. The method of claim 2, further comprising:
the collection being refined comprises a warehouse database; and
selecting the candidate polypeptides from the warehouse database based upon the at least partial amino acid sequence of the sample polypeptide.

5. A method of determining the primary structure of a sample polypeptide, comprising:
selecting a set of candidate polypeptides by the method of claim 1;
deriving a probability score of a match by comparing the absolute mass of the sample polypeptide with theoretical absolute mass data of candidate polypeptides; and
identifying the primary structure of the sample polypeptide based upon the greatest probability score of a match with one of the candidate polypeptides by ranking the probability scores of matches.

6. The method of claim 4, wherein the warehouse database further comprises at least one shotgun annotation of at least one polypeptide in the warehouse database.

7. The method of claim 6, wherein the shotgun annotation comprise a post-translational modification.

8. The method of claim 7, wherein said post-translational modifications comprise at least one member selected from the group consisting of ribosylation, phosphorylation, alkylation, hydroxylation, glycosylation, oxidation, reduction, myristylation, biotinylation, ubiquination, iodination, nitrosylation, amination, sulfur addition, cyclization, nucleotide addition, fatty acid addition, and acylation.

9. The method of claim 4, wherein the warehouse database is stored in the electronic memory of a computer.

10. The method of claim 9, wherein a user may retrieve information from the warehouse database through accessing the computer via electronic communication through a retrieval algorithm.

11. The method of claim 10, wherein the retreival algorithm further comprises an internet software application.

12. A method of screening a compound for inhibitory activity of an enzyme that post-translationally modifies a polypeptide substrate, comprising:
contacting the enzyme with the compound to form a pre-mixture; and
adding to the pre-mixture the polypeptide substrate to form a reaction mixture;
analyzing the polypeptide substrate using the method of claim 5.

13. The method of claim 12, further comprising the addition of a co-factor that catalyzes reactions with the enzyme, wherein the co-factor comprises at least one member selected from the group consisting of ATP, ADP, AMP, GTP, GDP, GMP, CTP, CDP, CMP, UTP, UDP and UMP.

14. The method of claim 12, wherein the enzyme is immobilized to a solid support.

15. A computer program product for use with a computer, the computer program product comprising a computer usable medium having computer readable program code in said medium for selecting a set of candidate polypeptides for a sample polypeptide, said computer program product, comprising:
computer readable program code for directing the computer to select a set of candidate polypeptides for a sample polypeptide, comprising:
a first refining of a collection of candidate polypeptides from differences in mass of fragments of the sample polypeptide produced by mass spectrometry; and
a second refining of the collection of candidate polypeptides from the absolute mass of the sample polypeptide and the absolute mass of the fragments.

16. The computer program of claim 15, wherein the computer readable program code for directing the computer to determine the first refining of the collection, wherein the first refining comprises determining at least a partial amino acid sequence of the sample polypeptide from the differences in mass of the fragments.

17. The computer program product of claim 16, further comprising computer readable program code for directing the computer to determine the absolute mass of an intact form of the sample polypeptide and the absolute mass of the fragments of the sample polypeptide.

18. The computer program product of claim 16, further comprising computer readable program code for dirccting the computer to the select the candidate polypeptides from a collection of protein forms based upon the at least partial amino acid sequence of the sample polypeptide.

19. The computer program product of claim 16, further comprising computer readable program code for directing the computer to select a set of candidate polypeptides by the method of claim 1, to derive a probability score of a match by comparing the absolute mass of the sample polypeptide with theoretical absolute mass data of candidate polypeptides; and to identify the primary structure of the sample polypeptide based upon the greatest probability score of a match with one of the candidate polypeptides by ranking the probability scores of matches.

20. The computer program product of claim 15, further comprising a system, wherein the system comprises:
a computer;
a warehouse database of protein forms; and
primary utilities.

21. The computer program product of claim 20, wherein the primary utilities comprise at least one member selected from the group consisting of a data management system, an ion predictor, a data reduction tool, and a graphical viewer interface tool.

22. The computer program product of claim 20, wherein the warehouse database further comprises shotgun annotations.

23. The computer program product of claim 20, wherein the warehouse database further comprises dynamic shotgun annotations.

24. The computer program product of claim 20, wherein the system further comprises a retrieval algorithm, wherein the retrieval algorithm comprises an absolute mass searching mode and a sequence tag searching mode.

25. The computer program product of claim 24, wherein the absolute mass searching mode further comprises a Δm searching mode.

26. The computer program product of claim 20, further comprising a mass spectrometer in communication with the computer.

27. The computer program product of claim 20, wherein the computer is in communication with a user through an internet software application.

28. The computer program product of claim 20, further comprising:
a computer;
a warehouse database of protein forms;
a retrieval algorithm for searching the warehouse database;
a data management system;
an ion predictor;
a data reduction tool; and
a graphical viewer interface tool.

29. A system for selecting a set of candidate polypeptides for a sample polypeptide, comprising:
means for a first refining of a collection of candidate polypeptides from differences in mass of fragments of the sample polypeptide produced by mass spectrometry;
means for a second refining of the collection of candidate polypeptides from the absolute mass of the sample polypeptide and fragments of the sample polypeptide produced by mass spectrometry; and
a computer.

30. The system of claim 29, wherein the computer is in communication with a mass spectrometer.

31. The system of claim 29, wherein the computer is in communication with a user through an internet software application.

32. A system for selecting a set of candidate polypeptides for a sample polypeptide, comprising:
the computer program product of claim 15; and
a computer.

33. The method of claim 1, further comprising a third refining of the collection from the absolute mass of the sample polypeptide and fragments of the sample polypeptide, wherein the third refining of the collection occurs prior to the first refining of the collection.
